SEQUENCE LISTING (6 sequences)

SEQ ID NO: 1 - DNA, 24 bp, Artificial
  Sense oligonucleotide for I-SceI recognition site insertion in BamHI of pa1TI-EGFP
gatcatagggataacagggtaata

SEQ ID NO: 2 - DNA, 24 bp, Artificial
  Antisense oligonucleotide for I-SceI recognition site insertion in BamHI of pa1TI-EGFP
gatctattaccctgttatccctat

SEQ ID NO: 3 - DNA, 33 bp, Artificial
  Sense primer for PCR amplification of mRFP1-polyA with addition of Bgl II sites
gaagatctcttaagcatggcctcctccgaggac

SEQ ID NO: 4 - DNA, 35 bp, Artificial
  Antisense primer for PCR amplification of mRFP1-polyA with addition of Blg II sites
cctagatctgctagcatacattgatgagtttggac

SEQ ID NO: 5 - DNA, 30 bp, Artificial
  Oligonucleotide for insertion of an I-CreI recognition site
ctgggttcaaaacgtcgtgagacagtttgg

SEQ ID NO: 6 - DNA, 29 bp, Artificial
  Oligonucleotide for insertion of an I-CeuI recognition site
cgtaactataacggtcctaaggtagcgaa

(19) United States
(12) Patent Application Publication
(10) Pub. No.: US 2008/0113437 A1
(43) Pub. Date: May 15, 2008

(54) In-vitro method for producing oocytes or eggs having targeted genomic modification

(75) Inventors: Jean-Stephane Joly, Versailles (FR); Violette Thermes, Paris (FR); Frederic Sohm, Gif-Sur-Yvette (FR)

Correspondence Address: HARNESS, DICKEY & PIERCE, P.L.C., P.O. BOX 828, BLOOMFIELD HILLS, MI, 48303, US

(73) Assignee: Institut National de la Recherche Agronomique-INRA, Paris Cedex (FR)

(21) Appl. No.: 11/818,517

(22) Filed: Jun. 14, 2007

Related U.S. Application Data
(63) Continuation of application No. PCT/FR05/03182, filed on Dec. 19, 2005.

(30) Foreign Application Priority Data
Dec. 17, 2004 (FR) 04/13521

(51) Int. Cl. C12N 15/87; C12N 9/18

(52) U.S. Cl. 435/463; 435/197

(57) ABSTRACT

The invention relates to an in vitro method for introducing a targeted genome modification into an oocyte or an egg and a method for performing a random insertion in the genome of a host cell.

### CROSS-REFERENCE TO RELATED APPLICATIONS

This application is a continuation of PCT Serial No. PCT/FR2005/003182, filed Dec. 19, 2005, which claims priority to French Application Serial No. 04/13521, filed Dec. 17, 2004, both of which are incorporated by reference herein.

### BACKGROUND AND SUMMARY

The invention concerns an in vitro method for introducing a targeted genome modification into an oocyte or an egg and a method for performing a random insertion in the genome of a host cell.

Transgenesis is a molecular genetic technique by which the exogenous DNA is introduced into the genome of a multicell organism and is transmitted to the descendants of the latter. This transmission to the descendants requires the stable integration of the DNA in the genome of the embryo, at an early stage of development.

At the present time, one of the most widely used transgenesis techniques is that of micro-injection of naked DNA into a mammal egg, which, in a certain number of cases, results in the integration of some of the DNA molecules micro-injected into the genome of the egg. Other techniques can be used for transgenesis, in particular the techniques of introducing exogenous DNA into a living cell, which are well known to persons skilled in the art, in particular electroporation, transfection using calcium phosphate precipitation, liposomes or modified lipids such as Lipofectamine® (INVITROGEN).

In the case of a targeted integration of an exogenous DNA into the genome, it is necessary to use the homologous recombination mechanism. In this case, the exogenous DNA must have nucleic acid sequences homologous with those present at the targeted integration site in the genome. However, these homologous recombination mechanisms operate at an extremely low frequency in the majority of organisms. Since recently, the use of endonucleases involved in yeast in the ‘intron homing’ mechanism, which belong to the family of ‘meganucleases,’ has made it possible to significantly increase these frequencies of homologous recombination in cell cultures and in particular in embryonic mammal strain cells (COHEN-TANNOUDJI et al., Mol. Cell. Biol., vol. 18(3), p:1444-1448, 1998). In these cells, the induction of the expression of an exogenous meganuclease gives rise to a double-strand break in the genomic DNA at a specific nucleic acid sequence of large size, 18 base pairs for the meganuclease I-SceI, followed by a homologous recombination between sequences of an exogenous DNA molecule and homologous sequences framing this break site. These meganucleases thus make it possible to replace or delete a sequence of interest in the genomic DNA or to introduce an exogenous sequence into the genomic DNA, and this in a ‘targeted’ fashion.

Surprisingly, the inventors revealed that it was possible, by introducing a sequence of exogenous nucleic acid and the meganuclease I-SceI into an egg, which has in its genome an I-SceI site framed by sequences homologous with the exogenous nucleic acid sequence, to obtain an egg having a targeted genome modification corresponding to the insertion by homologous recombination of an exogenous nucleic acid sequence at the genome I-SceI site.

The discovery of the inventors makes it possible to demonstrate that, if the homologous recombination mechanism that uses a meganuclease can be implemented in vivo, the mechanism can also be effected directly in oocytes or eggs with sufficient efficacy, and this without compromising the implementation of the programme of development of the organism. The method of the inventors then makes it possible to obtain an egg or oocyte having a targeted genome modification, and potentially to obtain directly a mature genetically modified organism having such a targeted genome modification, and this in all its cells. The targeted genome modification can then correspond to a deletion or insertion, in particular the insertion of a sequence mutated with respect to the wild sequence.

The cell of the egg or oocyte contains a large cytoplasm compared with that of a normal cell, which makes it difficult to access the nucleus that contains the genetic material. In addition, the presence of a membrane (the vitelline membrane) and of a chorion present specifically around the eggs in order to protect them, limits access to the cell. These barriers generally require the use of special techniques, such as direct injection into the cell. The complexity of the techniques that can be used limits the number of eggs that it is possible to treat to a few hundreds of eggs per experiment.

The feasibility of a targeted genome modification method at the level of the egg or oocyte therefore required the events causing a targeted genome modification to operate with sufficient frequency for a person skilled in the art to have a reasonable hope of success in implementing such a method.

Obviously such a reasonable hope of success did not exist prior to the implementation of the method according to the invention. This is because, in the absence of meganucleases, the homologous recombination mechanism is a very rare genetic process at the level of the egg. The frequency of such a homologous recombination is probably less than or equal to that observed in embryonic stem cells, that is to say approximately one event for one million cells. The use of meganucleases to increase the frequency of homologous recombination, in particular in embryonic stem cells (ES; COHEN-TANNOUDJI et al., 1998, aforesaid) also did not enable the person skilled in the art to have a reasonable hope of success. This is because, even if the frequency of homologous recombination is increased in this case, this at the very most reaches a frequency of 6×10⁻⁶, which obviously made the technique inapplicable to eggs.

The article by COHEN-TANNOUDJI et al. (1998, aforesaid) suggests on the contrary to a person skilled in the art the use of a homologous recombination method based on embryonic stem cells in culture, which have the advantage of being able to be obtained in large numbers and to make it possible to obtain transgenic animals after injecting, into an embryo at the blastocyte stage, rare cells benefiting from the targeted genome modification. However, the transgenic organism obtained is termed ‘mosaic’ since it has both cells derived from the initial embryo and genetically modified embryonic stem cells injected. It is then necessary to effect crossings between the animals obtained so as to obtain animals where all the cells are genetically modified. The method according to the invention makes it possible to directly obtain transgenic animals where all the cells have the targeted genome modification. In addition, and in the case of organisms where no lineage of embryonic stem cells has been isolated, the prior art suggested to a person skilled in the art the isolation of such cells and in no case the method according to the invention, which made it possible to obtain transgenic animals directly from the egg for such organisms.

The article by SEGAL and CAROLL (Proc. Natl. Acad. Sci. USA, vol. 92, p: 806-810, 1995) describes a homologous recombination mechanism in a Xenopus oocyte in the presence of I-SceI meganuclease. However, the homologous recombination is effected in a circular plasmid that has an I-SceI site whereas no site exists in the genomic DNA for the meganuclease. In addition, though this article shows the obtaining of homologous recombination in the plasmid, nothing made it possible to predict sufficient efficacy of the mechanism for applying it to genomic DNA. The plasmid was injected in very large quantities and simultaneously with the I-SceI meganuclease, which is unstable when it is not fixed to its site. This large quantity of I-SceI sites and this co-injection, which facilitated the stabilisation of the meganuclease, in no case made it possible to predict the frequency of homologous recombination obtained in the presence of a site that is rare since located at the very most at a few copies in the genomic DNA, and in addition is difficult to access because of the compact structure of the genomic DNA. It could legitimately be expected, given the structure of the chromatin and the rarity of the sites, for the meganuclease to obtain no access to one of its sites before it has been degraded.

Consequently, a first object of the invention corresponds to an in vitro method of producing oocytes or eggs of non-human vertebrates having a targeted genome modification comprising:
a) a step of expressing an endonuclease in the nucleus of an oocyte or egg, characterised in that the endonuclease is introduced exogenously and in that the genomic DNA of the egg or oocyte has at least one recognition site for the endonuclease, which recognition site corresponds to a specific sequence of nucleic acids of at least 12 base pairs, allowing:
   (i) the sequence-specific binding of the endonuclease at the recognition site,
   (ii) the consequent causing of a double-strand break in the genomic DNA by the endonuclease at the recognition site or in its adjoining regions, preferably within fewer than 100 base pairs of the recognition site, and then
   (iii) the repair of the double-strand break by a homologous recombination method; and
b) a step of identification of the eggs or oocytes having the targeted genome modification sought.

“Egg” means a single cell resulting from the fertilization of a female gamete by a male gamete which contains all the potentialities necessary for the formation of a new organism. More simply, the egg corresponds to an embryo at the single-cell stage.

“Oocyte” means a female reproductive cell obtained during a maturation phase of the ovogenesis.

Preferably, the method according to the invention is an in vitro method of producing eggs of non-human vertebrates having a targeted genome modification.

By way of example of non-human vertebrates where it is possible to use the eggs or oocytes in the method according to the invention, it is possible to cite mammals such as rodents, sheep, bovines or non-human primates, reptiles, amphibians such as the Xenopus, birds such as hens, insects such as flies and fish such as zebrafish or Medaka. Preferably, the egg or oocyte used in the method of the invention is a fish egg or oocyte, such as salmon, trout, tuna, halibut, catfish, zebrafish, Medaka, carp, stickleback, Astyanax, tilapia, redfish, bass, sturgeon or loach. In a particularly preferred manner the egg of oocyte used is an egg or oocyte of a zebrafish (Danio rerio) or Medaka (Orizias latipes). The method according to the invention also applies without difficulty to other aquatic species such as frog, Xenopus, shrimp and sea urchin.

“Recognition site” means a specific nucleic acid sequence which has a length of at least 12 base pairs to which a given endonuclease specifically binds and which allow, after the binding of the endonuclease to it, the causing of a double-strand break in the DNA by the endonuclease. Preferably the recognition site corresponds to a specific nucleic acid sequence of at least 16 base pairs, and in a particularly preferred manner at least 18 base pairs. “Specific nucleic acid sequence” means a DNA sequence, preferably a double-strand DNA sequence.

The identification step can be performed using techniques well known to persons skilled in the art. This identification step can use, by way of example, the Southern or PCR techniques, on the isolated genomic DNA from the egg or oocyte obtained, using a probe or specific initiators respectively. In the case where the targeted genome modification corresponds to an insertion of a sequence of exogenous nucleic acids comprising a reporter gene, the identification step can use techniques of detecting the activity of the reporter gene. Such detection techniques depend on the reporter gene used and are well known to persons skilled in the art. In the case where the targeted genome modification corresponds to an insertion of a sequence of exogenous nucleic acids comprising a selection gene, the identification step corresponds to a step of culturing the egg or oocyte in an adapted medium. The culture conditions for such a step depend on the selection gene used and are well known to persons skilled in the art.

According to a particular embodiment, the specific nucleic acid sequence for the endonuclease corresponds to the consensus binding sequence determined from the endonuclease or to a sequence derived from the consensus sequence. This is because some endonucleases are capable of binding to sequences not having a perfect identity with their consensus sequence and, following on from this binding, effecting a double-strand break at the latter or in its adjoining regions.

Advantageously, the specific sequence has more than 90% identity with the consensus sequence, preferably more than 95%, and particularly preferably more than 98% with the consensus sequence. “Percentage identity” means the percentage of nucleic acid with identical nature and position between the specific sequence and the consensus binding sequence determined for the endonuclease.

Depending on the endonuclease used, it is possible to obtain a double-strand break in the DNA either at the recognition site specifically or in the sequences adjoining the recognition site, preferably fewer than 100 base pairs the recognition site, preferentially fewer than 50 base pairs, and in a particularly preferred manner fewer than 20 base pairs. Advantageously, the double-strand break caused by the endonuclease is located at its recognition site in the genomic DNA. The recognition site can be present in the genomic DNA of wild individuals or it can be introduced into the genomic DNA by transgenesis.

According to a preferred embodiment of the present invention, the recognition site is introduced by transgenesis into the genomic DNA of the egg or oocyte. The introduction of this recognition site into the genomic DNA can be effected in a targeted fashion or randomly. Advantageously, the introduction by transgenesis is able to be effected either in the oocyte or egg used in the method according to the invention, or in an oocyte, egg or cell from which a sexually mature organism has been able to develop and from which the oocyte or egg used in the method according to the invention came.

According to a particular embodiment of the preferred embodiment, the introduction of the recognition site is effected in a targeted fashion. Such a targeted introduction can be effected by homologous recombination according to techniques well known to persons skilled in the art. By way of example, COHEN-TANNOUDJI et al. (1998, aforesaid) describes the injection into the nucleus of a vector that contains a selection gene and a recognition site for a meganuclease, which are framed by sequences of nucleic acids homologous with the target sequences of the genomic DNA. After selection of cells that have integrated the construction in a stable manner, in particular using an appropriate culture medium, the cells that integrated the construction at the required position in the genomic DNA are identified, in particular by Southern blot or by PCR. The recombination rates being low under these conditions, the number of cells having a targeted insertion is extremely small. Advantageously, the targeted introduction of the recognition site for the endonuclease in the genomic DNA is effected by homologous recombination.

According to a second particular embodiment of the preferred embodiment, the introduction of the recognition site is effected randomly. To this end, various techniques can be used. By way of example, CHOULIKA et al (1998, Mol. Cell. Biol., vol. 15(4), p: 1968-1973, 1995) describes the use of a retroviral vector for the integration of recognition sites for the I-SceI meganuclease in the genomic DNA. The PTC application WO 03/025183 describes another method of random integration of recognition sites for the I-SceI meganuclease in the genomic DNA of a fish egg by micro-injecting simultaneously into its nucleus the I-SceI meganuclease and a fragment of DNA that has a reporter gene framed by two I-SceI recognition sites. It is also possible to use the method of random integration of nucleic acid sequences according to the invention described in the examples. Advantageously, the random introduction of the recognition site for the endonuclease in the genomic DNA is effected by the method of random integration of nucleic acid sequences described in the patent application WO 03/025183 or by the method of random integration of nucleic acid sequences described in the examples.

Many endonucleases capable of binding to a specific sequence of at least 12 base pairs, of consecutively causing a double-strand break in the DNA at the specific sequence, or in its adjoining regions, and finally causing the repair of the double-strand break by a homologous recombination method are known to persons skilled in the art. By way of example of such endonucleases, meganucleases can be cited.

Meganucleases constitute a family of enzymes that effect a double-strand break in the DNA with very low frequency. This is because the meganucleases have recognition sites of 12 to 40 base pairs whereas conventional restriction enzymes have recognition sites generally of around 4 to 8 base pairs. The probability of the presence of such a recognition site in the genomic DNA is therefore extremely low. Meganucleases are also well characterised from a structural and mechanistic point of view. Meganucleases are divided into four distinct families on the basis of amino acid units conserved.

The dodecapeptide family (dodecamer, DOD, DOD, D1-D2, LAGLI6DADG, P1-P2) is the largest family with more than 150 sequences grouped together according to whether they have one (I-CeuI, I-CreI) or two copies (I-ChuI, I-CsmI, I-DmoI, I-PanI, I-SceI, I-SceII, I-SceIII, I-SceIV, F-SceI, F-SceII, PI-AaeI, PI-ApeI, PI-CeuI, PI-CirI, PI-CtrI, PI-DraI, PI-MavI, PI-MflI, PI-MgoI, PI-MjaI, PI-MkaI, PI-MleI, PI-MtuI, PI-MtuHI, PI-PabIII, PI-PfuI, PI-PhoI, PI-PkoI, PI-PspI, PI-RmaI, PI-SceI, PI-SspI, PI-TfuI, PI-TliI, PI-TliII, PI-TspI, PI-TspII, PI-BspI, PI-MchI, PI-MfaI, PI-MgaI, PI-MgaII, PI-MinI, PI-MmaI, PI-MshI, PI-MsmII, PI-MthI, PI-TagI, PI-ThyII) of a conserved motif of twelve amino acids, the dodecapeptide. The meganucleases with a dodecapeptide have a molecular mass of around 20 kDa and act in homodimer form. Meganucleases with two dodecapeptides have a molecular mass of 25 to 50 kDa, with 70 to 150 residues between both motifs, and are active in the form of monomers.

The GIG family has a complete conserved unit KSGIY-X_{10/11-}YIGS (I-NcrI, I-NcrI, I-PanII, I-TevI) or partial (I-TevII) and the enzymes in this family cut the DNA at a site different from their recognition site.

The HC family has sequences with high histidine and cystein contents (I-PpoI, I-DirI, I-HmuI, I-HmuII) with generally a conserved sequence that corresponds approximately to “SHLC-G-G-H-C.” The meganuclease best characterised for this family is the I-PpoI enzyme. The HNH family has a consensus sequence “HH-N-H-H” in a window of 35 residues (I-TevIII) and particular properties of cutting the DNA. However, various meganucleases have also been identified that have not been able to be associated with these four families. At the present time, these meganucleases are five in number and correspond to F-SceI, F-SceII (HO), F-SuvI, F-TevI and F-TevII. However, all these meganucleases are capable of inducing a double-strand break in the DNA having a recognition site, and this specifically at the latter or in its adjoining regions.

In addition, many meganucleases have a nuclear location signal (NLS). This protein sequence facilitates the entry of the meganuclease into the nucleus and thus the homologous recombination mediated by this. The I-SceI meganuclease constitutes an example of such a meganuclease. However, a person skilled in the art can construct a derived meganuclease having such a nuclear location signal, in the case where such a signal is absent from the wild meganuclease, and this according to techniques well known in molecular biology for producing recombinant proteins.

According to a second preferred embodiment of the method of the present invention, the endonuclease used is a meganuclease or an enzyme derived from such a meganuclease, which may be synthetic. By way of example of meganucleases, the following meganucleases can therefore be cited: I-CeuI, I-CreI, I-ChuI, I-CsmI, I-DmoI, I-PanI, I-SceI, I-SceII, I-SceIII, I-SceIV, F-SceI, F-SceII, PI-AaeI, PI-ApeI, PI-CeuI, PI-CirI, PI-CtrI, PI-DraI, PI-MavI, PI-MflI, PI-MgoI, PI-MjaI, PI-MkaI, PI-MleI, PI-MtuI, PI-MtuHI, PI-PabIII, PI-PfuI, PI-PhoI, PI-PkoI, PI-PspI, PI-RmaI, PI-SceI, PI-SspI, PI-TfuI, PI-TliI, PI-TliII, PI-TspI, PI-TspII, PI-BspI, PI-MchI, PI-MfaI, PI-MgaI, PI-MgaII, PI-MinI, PI-MmaI, PI-MshI, PI-MsmII, PI-MthI, PI-TagI, PI-ThyII, I-NcrI, I-NcrII, I-PanII, I-TevI, I-PpoI, I-DirI, I-HmuI, I-HmuII, I-TevII, I-TevIII, F-SceI, F-SceII (HO), F-SuvI, F-TevI and F-TevII or a meganuclease derived from one of them. The recognition sites and the specificities of these various meganucleases are well known to persons skilled in the art and are described in particular at the http website rebase.neb.com. Preferably the meganuclease is the meganuclease I-SceI described in the patent U.S. Pat. No. 6,238,924.

Derived meganuclease or enzyme derived from a meganuclease means a recombinant protein that has sequences of a wild meganuclease and that is capable of recognising a recognition site different from the wild meganuclease site and/or effecting a double-strand break in the DNA at a different position or according to a different mechanism from the wild meganuclease. The derived meganuclease also makes it possible to cause the repair of the double-strand break by a homologous recombination mechanism. By way of example of such derived meganucleases, it is possible to cite in particular recombinant meganucleases where the bonding domain of the DNA is derived from other proteins bonding to the DNA such as restriction endonucleases of the IIS type or transcription factors. By way of examples of such derived meganucleases, it is also possible to cite recombinant meganucleases that have one or more nuclear location sites absent from the wild meganucleases from which they are derived. The endonuclease can be introduced exogenously into the egg or oocyte in various forms, namely in the form of a protein or in the form of a sequence of nucleic acids permitting the expression of the endonuclease in the egg or oocyte.

According to a third preferred embodiment of the method according to the invention, the endonuclease is introduced into the egg or oocyte in the form of a protein. Techniques allowing the introduction of such an endonuclease in the form of a protein are known to persons skilled in the art. By way of example of such techniques, in particular micro-injection can be cited. Advantageously, the concentration of the endonuclease introduced exogenously is between 0.1 and 5 units per μl and per egg or oocyte, preferably between 0.5 and 2.5 units per μl, and particularly preferably between 1 and 2 units per μl. The volume injected per egg or oocyte forms part of the general knowledge of a person skilled in the art and is around 10% of the volume thereof. By way of example, the volume able to be injected into a zebrafish or Medaka egg or oocyte is between 300 pl and 1 nl. The quantity of nucleic acid introduced per egg or oocyte is then between 0.3×10⁻⁴ and 5×10⁻³ units, preferably between 1.5×10⁻⁴ and 2.5×10⁻³ units and particularly preferably between 0.3×10⁻³ and 2×10⁻³ units.

According to a fourth preferred embodiment of the method according to the present invention, the endonuclease is introduced in the form of a nucleic acid molecule allowing the expression of the endonuclease in the egg or oocyte. The nucleic acid molecule then comprises an open reading frame, coding for the endonuclease, under the control of regulation sequences allowing the expression of the endonuclease in the egg or oocyte. Nucleic acid molecule means both DNA and RNA molecules and hybrid DNA/RNA molecules, which can be in a single-strand or double-strand form. By way of example of a nucleic acid molecule that can be used in the method according to the invention, an RNA molecule coding for the endonuclease or an expression vector comprising an open reading frame coding for the nuclease can be cited.

Expression vector means a nucleic acid molecule capable of transporting and allowing the expression of a sequence of nucleic acids of interest, to which it is operably linked. Such an expression vector contains a promoter sequence allowing the expression of the endonuclease in the egg or oocyte. By way of example of such promoters, it is possible to cite in particular the promoter of α₁-tubulin or α-actin, or strong constituent promoters well known to persons skilled in the art such as the promoter of the cytomegalovirus (CMV). The expression vector can also contain other regulation sequences corresponding to a replication origin, a ribosome binding site, one or more splicing sites, a polyadenylation site or a transcription termination site.

An expression vector that can be used in the method according to the invention may correspond, non-limitingly, to a YAC (yeast artificial chromosome), to a BAC (bacterial artificial chromosome), to a viral vector, to a plasmid vector, to a phagemid, to a cosmid, to an RNA vector, to a vector derived from a baculovirus, a phage, a transposon or an RNA or a DNA molecule, linear or circular. Such vectors are well known to persons skilled in the art. Examples of viral vectors are in particular retroviruses, adenoviruses, parvoviruses, coronaviruses, orthomyxoviruses, rhabdoviruses, paramyxoviruses, picornaviruses, alphaviruses, adenoviruses, herpes viruses and pox viruses. The expression vector used is preferably a plasmid vector.

Techniques for introducing a nucleic acid molecule into an egg or oocyte are well known to persons skilled in the art. Examples of such techniques are micro-injection, electroporation, transfection by means of liposomes or modified lipids such as Lipofectamine®) (INVITROGEN), or by means of calcium phosphate precipitation. Preferably this introduction is effected by the micro-injection technique. The targeted genome modification introduced following the homologous recombination at the double-strand break site of the DNA may correspond either to a deletion of a genome sequence, in the case where the recombination takes place between two homologous sequences of the genomic DNA on each side of the break site, or to an insertion in the case where the recombination takes place, at homologous regions, between an exogenous nucleic acid sequence and the genomic DNA.

According to a fifth preferred embodiment of the method according to the invention, the method also comprises a step of introducing, into the oocyte or egg, an exogenous nucleic acid sequence that has homology with the nucleic acid sequences located upstream and downstream of the recognition site for the endonuclease that is present in the genomic DNA. Exogenous nucleic acid sequence means a double-strand DNA sequence that is introduced into an egg or oocyte, which can be in a linear or circular form. Preferably the exogenous nucleic acid sequence is in circular form. Advantageously, the concentration of the nucleic acid sequence administered by egg or oocyte is between 1 and 50 ng per μl, preferably between 5 and 40 ng per μl, and particularly preferably between 10 and 30 ng per μl. As seen previously, the volume injected by egg or oocyte forms part of the general knowledge of a person skilled in the art and is around 10% of the volume thereof. By way of example, the volume liable to be injected into an egg or oocyte of a zebrafish or Medaka is between 300 μl and 1 nl. The quantity of nucleic acids introduced by egg or oocyte is then between 0.3 and 50 μg, preferably between 15 and 40 pg and particularly preferably between 3 and 30 pg.

According to a particular embodiment of the preferred embodiment, the exogenous nucleic acid sequence is derived from the genome sequence, in particular a mutated form of the genome sequence or an isoform of the genome sequence which comes from another individual or another organism. In this case, the homologous recombination mechanism gives rise to an insertion of the exogenous nucleic acid sequence that is like a ‘replacement’ of the genome sequence.

According to a second particular embodiment of the preferred embodiment, the exogenous nucleic acid sequence comprises a nucleic acid sequence of interest that is framed by two distinct nucleic acid sequences, which have homology with the nucleic acid sequences located upstream and downstream respectively of the endonuclease's recognition site that is present in the genomic DNA. The nucleic acid sequence of interest may correspond to a gene or to a regulating sequence (such as a promoter or an activator) where it is wished to determine the associated activity and/or phenotype in a transgenic vertebrate obtained by the method according to the invention. The nucleic acid sequence of interest may comprise a reporter gene, such as beta-galactosidase, GFP (green fluorescent protein), RFP (red fluorescent protein), or a selection gene, such as neomycin phosphotransferase, hygromycin phosphotransferase, histidinol dehydrogenase or thymidine kinase. The use of such a selection reporter gene may facilitate the identification of the eggs or oocytes having the targeted genome modification sought. Preferably, the reporter or selection gene does not comprise associated promoter sequences so as to identify the eggs or oocytes having the expected expression profile corresponding to the targeted genome modification sought. Advantageously, the nucleic acid sequence or sequences, which have homology with the nucleic acid sequences located upstream and downstream of the recognition site for the endonuclease, have a length of at least 50 base pairs, preferably at least 100 base pairs, and particularly preferably at least 250 base pairs. The sequences may have a greater size, however a size of more than 1,000 base pairs does not increase the efficacy of the homologous recombination.

Advantageously again, the homologous nucleic acid sequence or sequences have an identity of sequence of at least 80% with the nucleic acid sequences located upstream and downstream of the endonuclease's recognition site in the genomic DNA, preferably an identity of at least 90%, and particularly preferably an identity of at least 95%. The identity between two nucleic acid sequences corresponds to the percentage of identical nucleotides located at an identical position between two nucleic acid sequences. Many programmes or algorithms make it possible to calculate identity percentages, including FASTA or BLAST. These programmes are in particular available on the http website of NCBI (National Centre for Biotechnology Information) ncbi.nim.nih.gov/. Preferably, the homology is determined by the BLAST programme and particularly preferably with a BLAST programme using the BLOSUM62 matrix.

Advantageously, the exogenous nucleic acid sequence is a vector. Vector means a nucleic acid sequence capable of transporting a nucleic acid sequence of interest to which it is linked. By way of example of vectors that can be used in the method according to the invention, it is possible to cite, non-limitingly, a YAC (yeast artificial chromosome), a BAC (bacterial artificial chromosome), or a suitable viral vector, such as an adenovirus, a plasmid vector, a phagemid or a cosmid. Preferably the vector used is a plasmid vector. Advantageously, the exogenous nucleic acid sequence has no recognition site for endonuclease.

Techniques for introducing a nucleic acid sequence into an egg or oocyte are well known to persons skilled in the art. By way of example of such techniques, it is possible to cite micro-injection, electroporation, transfection by means of liposomes or modified lipids such as Lipofectamine® (INVITROGEN), or using calcium phosphate precipitation. Preferably this introduction is effected by the micro-injection technique. The introduction of the exogenous nucleic sequence can be made in a deferred fashion or simultaneously with respect to that of the endonuclease or the nucleic acid molecule allowing the expression of the endonuclease. Preferably their introduction is simultaneous.

According to a sixth preferred embodiment of the method according to the invention, the method according to the invention also comprises a step of culturing the previously fertilised oocyte or the egg having a targeted genome modification under suitable conditions for allowing the development of the non-human vertebrate. Advantageously, the culture conditions used allow the development to full term of the non-human vertebrate. These culture conditions, like the oocyte fertilisation techniques, are well known to persons skilled in the art and depend on the organism used in the method of the invention. By way of example and for zebrafish or Medaka eggs, this culture step corresponds to the incubation of the eggs at a temperature of around 28° C., plus or minus 1 or 2° C.

According to a seventh preferred embodiment of the method according to the invention, the method also comprises a step prior to the culture step which corresponds to an incubation of the egg at a temperature less than the culture temperature by 5° to 20° C., preferably 10° to 15° C., and for a time making it possible to maintain a viability of the eggs greater than 5%, that is to say the number of eggs surviving as far as hatching, preferably greater than 10%, and particularly preferably greater than 15%. The maximum time during which the eggs or oocytes can be maintained can be determined simply by a person skilled in the art and depends on the resistance to temperature of the eggs or oocytes used.

Advantageously, such an incubation is carried out for a time between 1 and 24 hours, preferably between 1 and 20 hours, and particularly preferably between 1 and 10 hours. By way of example and in the case of Medaka eggs, this prior step corresponds to an incubation at a temperature of between 10° and 25° C., preferably between 12° and 19° C. and particularly preferably between 13° and 18° C. Advantageously, the step of identification of the eggs or oocytes having the targeted genome modification sought is performed on cells issuing from a non-human vertebrate organism obtained during the development of the eggs or oocytes after fertilization, preferably on cells issuing from the mature non-human vertebrate organism.

### BRIEF DESCRIPTION OF DRAWINGS

FIG. 1 presents ³²P radiographic images of Southern-blots of NotI- and SacI-digested genomic DNA from F1 adult transgenic Medaka fish of three different lineages. For lineage F0.19, intense bands are evident at about 2 Kb (SacI), 4 Kb (NotI and SacI), and as indicated by a star (NotI); and a fainter band can be seen at about 3 Kb (SacI). For lineages F0.25-1 and F0.25-2, faint bands can be seen at about 4 Kb (SacI), as indicated by an arrow.

FIG. 2 presents schematic maps of possible pα1-EGFP-I transgene insertions and their orientations: direct tandem insert (2A); reverse tandem type I insert (2B); and reverse tandem type II insert (2C). The dashed line shows the approximate position of the ³²P-labeled specific probe.

FIG. 3 presents an analysis of 500 bp fragments of genomic DNA from adult transgenic Medaka fish, obtained by PCR amplification of I-SceI-recognition-site-containing loci, according to the scheme of FIG. 3A. Following digestion with I-SceI, the products were analyzed by gel electrophoresis, as shown in FIG. 3B.

### DETAILED DESCRIPTION

The following examples are provided by way of illustration and do not limit the extent of the present invention. Other advantages and characteristics of the invention will emerge in the light of the following examples.

### Example 1

### Random Insertion of an I-SceI Site in the Medaka Genome

1) pα₁TI-EGFP-I construction: The pa₁TI-GFP-I construction was obtained by inserting, in the plasmid pα₁TI-EGFP (Goldman et al., Transgenic Res., vol. 10(1), p: 21-33, 2001; HIEBER et al., J. Neurobiol., vol. 37(3), p: 429-440, 1998)), a recognition site for the I-SceI meganuclease between the promoter of the α_{I}-tubulin of the zebrafish and the reporter gene of the EGFP (enhanced green fluorescent protein).

In a first step, the pα₁TI-EGFP construction was digested by the enzyme BamHI (BIOLARGE) and the digested construction was then purified. The pa₁TI-EGFP construction digested by BamHI was then dephosphorylised and then purified again. Finally, a ligation reaction was performed between the pα₁TI-EGFP construction, digested by BamHI and dephosphorylised, and a double-strand oligonucleotide containing the site I-SceI site (in bold characters) and cohesive free ends, compatible with the digested BamHI site (sense oligonucleotide) (SEQ ID NO:1: 5′-GATCATAGGGATAACAGGGTAATA-3′); anti-sense oligonucleotide (SEQ ID NO:2: 5′-GATCTATTACCCTGTTATCCCTT-3′). The insertion and orientation of the recognition site for I-SceI at the BamHI site, between the promoting sequence of α₁-tubulin and that of the open reading frame of EGFP, and the conservation of the reading frame, were controlled by sequencing.

2) pact-GFPI2 construction: The pact-GFPI2 construction was obtained as described in THERMES et al. (Mechanisms of Development, vol. 118, p: 91-98, 2002). The insertion and orientation of the two functional recognition sites for the I-SceI meganuclease, upstream of the promoter of the α-actin of the zebrafish and downstream of the reporter gene of GFP (Green Fluorescent Protein) respectively, was controlled by sequencing.

3) Linearization of the pα₁TI-EGFP-I and pact-GFPI2 constructions: The transgene α₁TI-EGFP-I in linearised form was obtained by digestion of the pα₁TI-EGFP-I construction by the enzymes XhoI and AflII (BIOLABS). The fragment XhoI-AflII containing the transgene was then purified on a QIAEX II® column (QIAGEN), and then filtered on an Elutip-D® column (SCHLEICHER AND SCHUELL).

The transgene act-GFPI2 in linearised form was obtained by digestion of the pact-GFPI2 construction by the I-SceI meganuclease (ROCHE DIAGNOSTICS). The I-sceI-I--SceI fragment containing the transgene was then purified as previously.

4) Micro-injection of the transgenes and of the I-SceI meganuclease: Various DNAs were injected, either with or without I-SceI meganuclease, in a Medaka egg at the single-cell stage according to the protocol described in Thermes et al. (2002, aforesaid).

In the experiments carried out, the α₁TI-EGFP-I transgene was injected in linear form (fragment XhoI-AflII) and the act-GFPI2 transgene in linear form (fragment ISceI-I-SceI) or circular form (pact-GFPI2).

5) Expression of the transgene in the egg (F0): In order to monitor the expression of the transgenes in the micro-injected eggs, the fluorescence of the embryos was observed under a Leica MZFLIII microscope equipped with a UV lamp (excitation at 370-420 nm) and an emission filter at 455 nm for GFP.

In preliminary experiments in which plasmid pα₁TI-EGFP alone, and in circular form, was micro-injected into the egg at the single-cell stage, the results showed that the fluorescence of the EGFP is detectable in the NHC during the neuro-genesis and up to hatching (9 days post fertilisation st.39). More precisely, these experiments on transient expression of the pα₁TI-EGFP construction revealed that the promoter of α₁-tubulin of zebrafish is activated at the level of the central nervous system in the Medaka, principally in cells in the course of proliferation. The specific activity of the construction therefore appears to be similar to that described in zebrafish in GOLDMAN et al. (2001, aforesaid).

For the transgene α₁TI-EGFP-I, the results showed an expression profile similar to that of the pα₁TI-EGFP construction in the egg. The expression profile of the transgene act-GFPI2 is for its part similar to that observed in Thermes et al. (2002, aforesaid).

The proportion of embryos expressing the trensgenes in the various micro-injection experiments is described in Table 1 below.

TABLE I

| | | | Negative | Positive |
|---|---|---|---|---|
| DNA | | I-Scel | expression | expression |
| transgene | form | meganuclease | (%) | (%) |
| act-GFPI2 | linear | − | 49 | 51 |
| act-GFPI2 | circular | − | 50 | 50 |
| act-GFPI2 | circular | + | 16 | 84 |
| α₁TI-EGFP-I | linear | + | 10 | 90 |

In the absence of meganuclease, it is observed that close to 50% of the embryos that survive the micro-injection do not exhibit any fluorescence. On the other hand, when the various transgenes are micro-injected in the presence of meganuclease, a significant increase in the proportion of embryos expressing GFP is observed. Thus the result showed that, in the presence of I-SceI meganuclease, the negative embryos for the expression of the transgene α₁TI-EGFP-I represented approximately 10% of all the embryos injected (12%, n=116), that it is to say a proportion lower than that obtained with the transgene act-GFPI2 (16%). The transient expression of the transgene at generation F0 is therefore greatly improved by the co-injection of the meganuclease.

6) Transmission of the transgene to the descendants: The fish of generation F0 expressing the transgene were selected as a potential founder. These were raised to sexual maturity and were then crossed with wild partners. The embryos of the descendants (F1) were analysed for their expression of transgenes. The results are presented in table II below.

TABLE II

| | | | | Mean | Stand- |
|---|---|---|---|---|---|
| | | | Amount of | transmission | ard |
| DNA | | | transgenic F0 | levels in | error |
| Transgene | Form | I-Scel | (founders in %) | F1 (%) | Sm (%) |
| act-GFPI2 | linear | − | 5.9 (2/35) | 15.1 | 17.7 |
| act-GFPI2 | circular | − | 15.6 (5/32) | 17.6 | 22.2 |
| act-GFPI2 | circular | + | 30.5 (11/36) | 48.4 | 9.1 |
| α₁TI-EGFP-I | linear | − | 0 (0/17) | | |
| α₁TI-EGFP-I | linear | + | 21 (4/19) | 30 | 12.5 |

The results show that, in the case of control injections, (without co-injection with I-SceI), the majority of fish tested were not positive for GFP. This implies that the transgene was absent from the cells in the reproductive lineage in F0.

In the case of the transgene act-GFPI2, which is framed by two I-SceI recognition sites, it is observed that close to 30% of the individuals have integrated the transgene in a stable fashion in their genome. The mechanism proposed corresponds to the one described in THERMES et al. (2002, aforesaid) according to which the meganuclease allowed the integration in the genome of a transgene framed by the “two” recognition sites for it. According to the model proposed, the transgene would be excised by the meganuclease, which would protect the free ends and then allow the random integration of the excised transgene in the genome.

Surprisingly, it is observed that, among the 19 embryos co-injected with the transgene α₁TI-EGFP-I and I-SceI, and tested for transmission, four (that is to say 20%, n=19) generated descendants expressing GFP. It therefore seems that the model proposed in THERMES et al. (2002, aforesaid) does not take account of the mechanism of insertion of the transgene in the presence of the meganuclease since the presence of a single recognition site for this, which is not located in the immediate vicinity of the extremity, also allows stable integration of the transgene in the genome with a high frequency.

The 4 founding individuals obtained by the integration of the transgene α₁TI-EGFP-I were called F0.191, F0.251, F0.341 and F0.361. The F1 individuals issuing from these founders had a uniform green fluorescence in the CNS, then observable from the start of neurogenesis, at the early-neurula stage (25 hpf, st.17). In the case of the founder F0.251, the F1 individuals issuing from this had several levels of expression of GFP. It was possible to distinguish, in increasing order of fluorescence, individuals expressing GFP weakly, moderately or strongly (called F0.25-1, F0.25-2 and F0.25-3, respectively). Such variations in F1 may be due to the segregation of different concatemers of the transgene integrated in the genome, in F0, in three genetically distinct integration sites. These embryos (F1) were raised until hatching and only those slightly and moderately fluorescent were hatched and gave birth to sexually mature adult fish. The transmission of the transgene to the following generations (i.e. F2, F3 and F4) remained uniform, which confirms the stable integration of the transgene.

The mean rate of transmission of the founding individuals was estimated by following the expression of the transgenes in the descendants of the positive F1 individuals. The rates obtained were very variable and below 50%. In the case of the transgene α₁TI-EGFP-I, the mean was 30±12.5%. Only one of the four founding fishes (F0.19I) then significantly reached 50% of transmission, which corresponds to the percentage of a hemizygote transmission. In this case, it is probable that the transgene was integrated at F0 at a single site and in all the cells of the germ line. Finally, the results show an appreciable improvement in the efficacy of integration of the transgene in the germ line in the presence of two or only one I-SceI site.

7) Analysis of the integration of the transgene in the genome: The genome DNA was extracted from F1 adult transgenic fish, using proteinase K and phenol in accordance with the protocol described in SAMBROOK et al. (CSH Laboratory Press, Cold Spring Harbor, 1989).

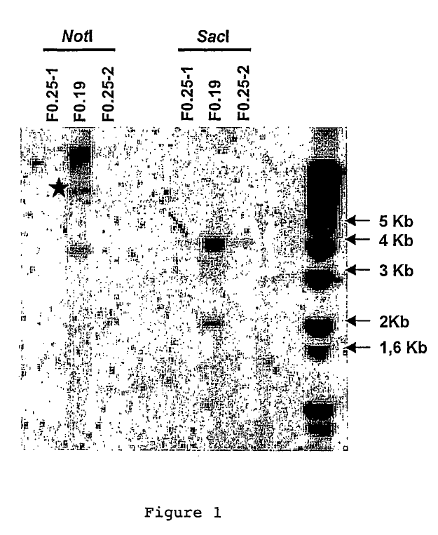

7-A. transgene α₁TI-EGFP-I: For the Southern blot experiments with the transgene α₁TI-EGFP-I, the genomic DNA was digested by SacI or NotI, separated on a 0.8% agarose gel (TAE 1×) and transferred by capillarity onto a nitrocellulose membrane, which was hybridised with a specific probe randomly marked with radioactive nucleotides (³²P). This probe corresponds to the sequence of the EGFP. The hybridisation at the transgene was revealed with a phosphor-imager after several hours of exposure to a silver film. The results are presented in FIG. 1.

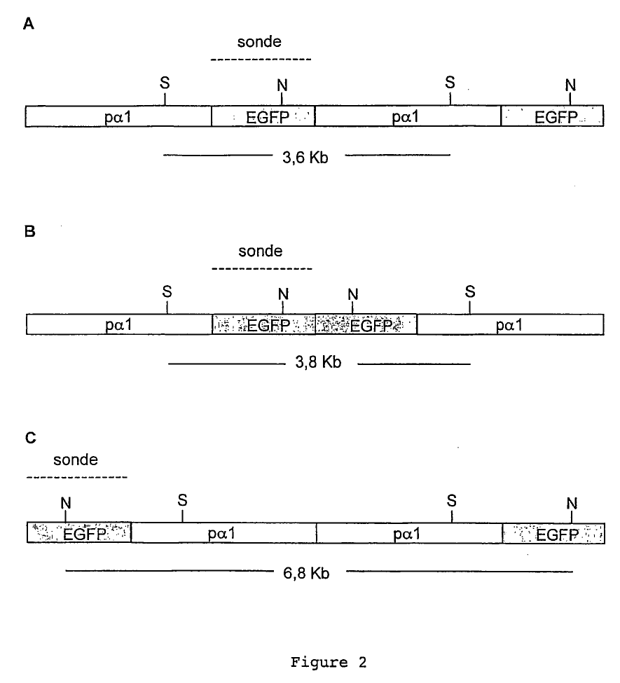

For the lineage F0.19, the digestion by SacI (site present at the exterior of the region recognised by the probe) reveals the presence of two intense bands with sizes close to 4 kb and several bands of lower sizes (2 kb and approximately 3 kb). The two bands of approximately 4 kb correspond probably to insertions of the transgene in direct tandem and in reverse tandem of type I respectively (FIGS. 2A and 2B respectively). For the bands of lower size (2 kb and 3 kb), these very probably correspond to fragments of junction between the integrated DNA and the genome DNA. The intensity of the two 4 kb bands, compared with the junction fragments, indicates the presence of these two types of tandem in large numbers in the genome and in comparable proportions.

For the lineages F0.25-1 and -2, the results show the presence of only one of the two diagnostic bands at 4 kb (FIG. 1, arrow). The comparison with the F0.19 profile indicates that it is probably a case of a form of concatemer in reverse tandem of type I (FIG. 2B). The low intensity of these bands suggest the presence of a small number of copies of the transgene in these lineages. These results have been confirmed for these three lineages by AgeI digestions (external to the region recognised by the probe; data not shown) and BamHI (no sites in the transgenes; data not shown).

The use of the NotI digestion makes it possible to analyse the lineages for the presence of the reverse tandem form of type II (FIG. 2C). The results show that only the lineage F0.19 has such integrations (FIG. 1, star).

Analysis of the genomic DNA of the lineage F0.36 revealed a digestion profile equivalent to that of the DNA of the lineage F0.19 (data not shown).

Consequently the two lineages F0.25 have an insertion profile with simple concatemers and in a small number of copies (reverse tandems of type I).

7-B. transgene act-GFPI2: The analysis of the transgenic lineages obtained with this transgene is described in THERMES et al. (2002, aforesaid).

The results show there are also insertion profiles with a small number of copies of the transgene (from one to eight copies) in the genomic DNA of the various lineages tested.

In conclusion, the use of the I-SceI meganuclease and constructions having one or two recognition sites for this makes it possible to obtain, in the majority of the lineages analysed, a reporter gene integrated in a stable fashion in the genome in the form of a single copy or a small number of copies. These two random insertion techniques therefore constitute prime techniques compared with the techniques normally used in transgenesis in which insertions are observed in large numbers in the genome (HACKETT, Biochemistry and Molecular Biology of Fishes, Elsevier, p: 207-240, 1993; IYENGAR et al, Transgenic Res., vol. 5, p: 147-166, 1996).

8) Integrity of the integrated I-SceI sites: In the case where it is envisaged using the transgenic lineages obtained in order to effect homologous recombination with the I-SceI meganuclease, and permit in particular the targeted integration of a gene of interest, it is important for the I-SceI site or sites integrated in the genome to be functional.

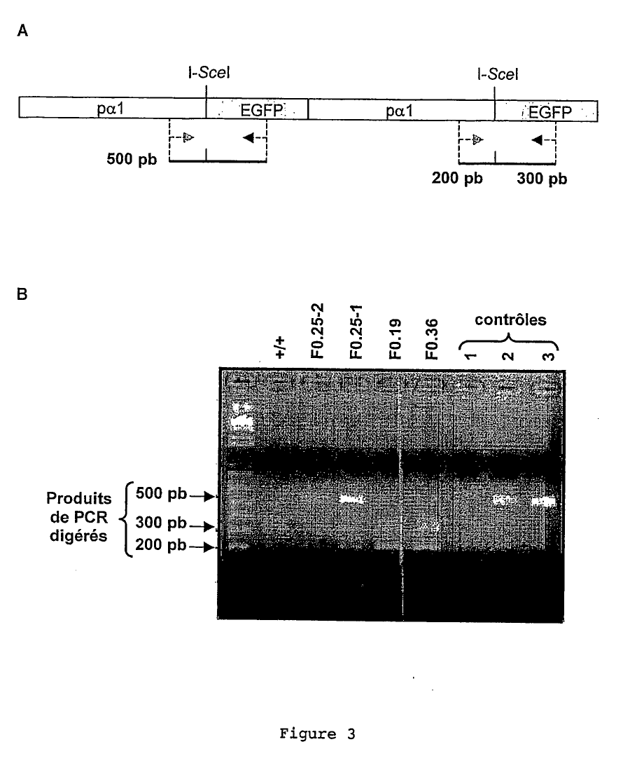

In order to test in vitro the integrity of the I-SceI sites integrated in the genome of the lineages F0.25-1, -2, F0.19 and F0.36, the genomic DNA of these lineages was purified. The genomic DNA was then amplified by PCR using specific initiators positioned on each side of the recognition site for I-SceI (FIG. 3A). The amplified DNA fragment has a length of 500 base pairs. The PCR products obtained were then digested by the enzyme I-SceI (ROCHE DIAGNOSTICS) according to the instructions of the manufacturer, and finally deposited on an electrophoresis gel. The results are presented in FIG. 3B.

The migration onto gel of the products of the reaction reveal the presence of a band at approximately 500 bp, corresponding to the uncut DNA, and two other bands of lower size 200 and 300 bp (cut DNA, FIGS. 3A and 3B). The presence of the band at 500 bp may result from an incomplete enzyme digestion or from the presence of mutated I-SceI sites. In all cases, the results show that the four lineages analysed possess non-mutated functional I-SceI sites, which are variable in number according to the lineage analysed.

### Example 2

### Targeted Insertion of a Transgene in the Genome of a Transgenic Medaka Lineage Having an I-SceI Site

1) Repair construction (RC): For the purpose of integrating a transgene in the Medaka genome in a targeted fashion, we tested a breach repair technique. For this, we used a second transgene containing the tracer gene of mRFP₁ (monomeric red fluorescent protein) surrounded on each side by sequences of at least 500 bp perfectly homologous with the regions surrounding the I-SceI site of the α₁TI-EGFP-I transgene (RC, Repair Construction). The homologous region at 5′ corresponds to the intronic sequence of the promoter α₁TI, which thus removes any possibility of expression of the mRPF₁ in episomal form.

In order to achieve this construction, the SacI-NotI fragment of the plasmid P₁TI-EGFP (1.7 kb), corresponding to the homology regions situated on each side of the I-SceI site, was purified and cloned in the pCRII-TOPO® vector (Invitrogen) linearised by a SacI-NotI digestion. The RH plasmid obtained was controlled by sequencing.

In parallel, the fragment BamHI-EcoRI of the plasmid mRFP₁-pRSETB (Campbell et al., Proc. Natl. Acad. Sci. USA, vol. 99(12), p: 7877-82, 2002), corresponding to the ORF of the mRFP₁, was subcloned in the plasmid p₁TI-EGFP, between the BamHI and NotI sites, in place of the EGFP. The construction obtained served as a substrate for amplifying the sequence mRFP₁-polyA by PCR by adding thereto a BglII site at each end (sense primer (SEQ ID NO:3: 5′-GAAGATCTCTTAAGCATGGCCTCCTCCGAGGAC-3′); anti-sense initiation (SEQ ID NO:4: 5′-CCTAGATCTGCTAGCATACATTGATGAGTTTG GAC-3′). The PCR product obtained was cloned in the plasmid pCRII-TOPO® (Invitrogen) and the sequence was controlled by sequencing. The fragment mRFP₁-polyA was then isolated by effecting a BglII digestion of this construction.

Finally, the repair construction (RC) was generated by introducing the fragment mRFP₁-polyA (BglII digestion) at the BamHI site of the RH construction. A controlled construction pα₁TI-mRFP1-EGFP was generated by introducing the same mRFP₁-polyA fragment into the p₁TI-EGFP construction.

2) Micro-injection of the repair construction and of the I-SceI meganuclease: The repair construction was co-injected in circular form with I-SceI meganuclease into a Medaka egg at the single-cell stage according to the protocol described in Thermes et al. (2002, aforesaid). The eggs used came from transgenic fish lineages (F3, F0.25-1 and -2, F0.19 and F0.36).

The repair construction (RC) was injected in circular form and at a final concentration of approximately 10 ng/μl, after amplification with a Midiprep® kit (Qiagen) and filtration (0.2 μm filter), in the presence of I-SceI at a concentration of one unit per μl.

After injection of the Medaka eggs at the single-cell stage, the eggs were placed in an incubator at 28° C. until hatching.

Simultaneously, and in order to verify the current expression of the mRFP1, the control construction pα₁TI-mRFP1-EGFP was injected alone into a Medaka egg at the single-cell stage according to the same protocol. The injection of this resulted in good expression of the mRFP₁ in the embryo, without any traces of green fluorescence.

3) Expression of the mRFP1 in the egg (F0): To monitor the expression of the transgenes in the micro-injected eggs, the fluorescence of the embryos was observed under a LEICA MZFLIII microscope equipped with a U.V. Lamp (excitation at 580-590 nm) and an emission filter at 607 nm for the mRFP1.

The injected embryos were observed under a microscope for their green and red fluorescence (in particular), around stages 28 (30 somites, 64 hpf) and 32 (end of somitogenesis, 4 days post-fertilisation). These experiments made it possible to isolate a positive mRFP₁ embryo issuing from the lineage F0.25-1 (weak expression of the GFP and integration of the transgene in the form of simple concatemers). Red fluorescence is detected solely in the CNS: it is weak but uniform, suggesting an equal distribution of the transgene in the first blastomeres. Since (i) this expression profile corresponds to the one obtained with the construction pα₁TI-mRFP1-EGFP and (ii) the distribution construction does not have a functional α₁ tubulin promoter, it is possible to conclude that the mRFP1 gene was integrated specifically, and by homologous recombination, upstream of the al tubulin promoter and at at least one of the I-SceI sites previously integrated in the genome of the F0.25-1 lineage.

The results therefore show that it is possible to obtain the specific insertion (by homologous recombination) of a transgene co-injected with a meganuclease in the genome of a Medaka egg which has a few copies of a recognition site for such a meganuclease.

4) Modifications of the micro-injection conditions: From the conditions described at 2), various modifications to the protocol were tested either independently of one another or together. The list of modifications tested is as follows:
Increase in the repair construction quantity up to a maximum of 50 ng/ml;
Increase in the quantity of meganuclease up to a maximum of 2 units per μl;
Reduction in the incubation temperature of the eggs after injection to a minimum of 13° C. and for a period varying from 2 to 16 hours before placing at 28° C.

The expression of the mRFP was monitored in the embryos obtained as described previously. The results are presented in table III.

TABLE III

| | Series | | | | | | | |
|---|---|---|---|---|---|---|---|---|
| | 1 | 2 | 3 | 4 | 5 | 6 | 7 | 8 |
| I-Scel | 1 | 1 | 1 | 2 | 2 | 2 | 2 | 2 |
| (U/μl) | | | | | | | | |
| Incubation | 28° C. | 28° C. | 28° C. | 28° C. | 28° C. | 18° C. | 18° C. | 13° C. |
| temperature | | | | | | (4 h) | (16 h) | (4 h) |
| (time) | | | | | | | | |
| DNA (ng/μl) | 12 | 25 | 50 | 25 | 50 | 25 | 25 | 25 |
| No of eggs | 442 | 1108 | 92 | 362 | 253 | 161 | 127 | 212 |
| injected | | | | | | | | |
| Survival after | 369 | 941 | 61 | 258 | 80 | 63 | 15 | 35 |
| 24 h | | | | | | | | |
| Hatching (%) | Nd | ~90 | ~90 | ~90 | Nd | Nd | 1 | 16 |
| Positive GFPs | 180 | 465 | 32 | 104 | 37 | 32 | Nd | 15 |
| Positive GFPs | 50.14 | 49.42 | 52.46 | 40.31 | 46.25 | 50.79 | Nd | 42.86 |
| in % | | | | | | | | |
| Positive RFPs | 0 | 2 | 0 | 1 | 1 | 5 | 0 | 12 |
| Positive RFPs | 0 | 0.21 | 0 | 0.39 | 1.25 | 7.94 | 0 | 34.29 |
| in % | | | | | | | | |
| Positive RFPs | 0 | 2(2) | 0 | 1 | 0 | 3(2) | 0 | 6(4) |
| hatched (viable) | | | | | | | | |
| Global efficacy | 0 | 0.18 | 0 | 0.28 | 0 | 1.86 | 0 | 2.83 |
| in % | | | | | | | | |

The results show that the most favourable conditions for obtaining a specific insertion are 2 units/ml of I-SceI meganuclease and 25 ng/ml of DNA. In addition, the results also show that the incubation of the eggs at a temperature of less than 28° C. following on from the micro-injection makes it possible to drastically increase the number of individuals having a marking (close to 3% for an incubation of 4 hours at 13° C. following the micro-injection).

### Example 3

### Targeted Insertion of a Transgene in the Genome of a Medaka Transgenic Lineage Using Other Meganucleases

Firstly random integrations are performed according to the protocol described in example 1, but with the I-CreI and I-CeuI meganucleases and constructions comprising respectively a recognition site for I-CreI meganuclease (SEQ ID NO:5: 5′-CTGGGTTCAAAACGTCGTGAGACAGTTTG G-3′) and I-CeuI (SEQ ID NO:6: 5′-CGTAACTATMCGGTCCTMGGTAGCGAA-3′) between the zebrafish α₁-tubulin promoter and the EGFP reporter gene.

The constructions and the protocol used for performing the targeted insertion are the same as previously described in example 2 but using the I-CreI and I-CeuI meganucleases (NEW ENGLAND BIOLABS).

1) In-vitro method of producing oocytes or eggs of non-human vertebrates having a targeted genome modification comprising:
a) a step of expressing an endonuclease in the nucleus of an oocyte or egg, characterised in that the said endonuclease is introduced exogenously and in that the genome DNA of the said egg or oocyte has at least one recognition site for the said endonuclease, which recognition site corresponds to a specific sequence of at least 12 pairs of bases, allowing:
(i) the sequence-specific fixing of the endonuclease at the said recognition site,
(ii) the consequent causing of a double-strand break in the genomic DNA by the said endonuclease at the said recognition site or in its adjoining regions, preferably at fewer than 100 pairs of bases of the said recognition site, and then
(iii) the repair of the said double-strand break by a homologous recombination mechanism; and
b) a step of identification of the eggs or oocytes having the targeted genome modification sought.

2) Method according to claim 1, characterised in that the said recognition site present in the genomic DNA was introduced by transgenesis.

3) Method according to either one of claims 1 or 2, characterised in that the said endonuclease is a meganuclease.

4) Method according to claim 3, characterised in that the meganuclease is chosen from the group comprising I-CeuI, I-CreI, I-ChuI, I-CsmI, I-DmoI, I-PanI, I-SceI, I-SceII, I-SceIII, I-SceIV, F-SceI, F-SceII, PI-AaeI, PI-ApeI, PI-CeuI, PI-CirI, PI-CtrI, PI-DraI, PI-MavI, PI-MflI, PI-MgoI, PI-MjaI, PI-MkaI, PI-MleI, PI-MtuI, PI-MtuHI, PI-PabIII, PI-PfuI, PI-PhoI, PI-PkoI, PI-PspI, PI-RmaI, PI-SceI, PI-SspI, PI-TfuI, PI-TliI, PI-TliII, PI-TspI, PI-TspII, PI-BspI, PI-MchI, PI-MfaI, PI-MgaI, PI-MgaII, PI-MinI, PI-MmaI, PI-MshI, PI-MsmII, PI-MthI, PI-TagI, PI-ThyII, I-NcrI, I-NcrII, I-PanII, I-TevI, I-PopI, I-DirI, I-HmuI, I-HmuII, I-TevII, I-TevIII, F-SceI, F-SceII (HO), F-SuvI, F-TevI and F-TevII or a meganuclease derived from one of them.

5) Method according to any one of claims 1 to 4, characterised in that the concentration of the said endonuclease introduced by egg or oocyte exogenously and in the form of protein is between 0.1 and 5 units per μl, preferably between 0.5 and 2.5 units per μl.

6) Method according to any one of the preceding claims, characterised in that the targeted genome modification at the break site corresponds to a deletion or insertion.

7) Method according to claim 6, characterised in that it also comprises a step of introducing, in the oocyte or egg, an exogenous nucleic acid sequence that has homology with the nucleic acid sequences located upstream and downstream of the recognition site for the endonuclease present in the genomic DNA.

8) Method according to claim 7, characterised in that the exogenous nucleic acid sequence introduced has no recognition site for the said endonuclease.

9) Method according to either one of claims 7 or 8, characterised in that the concentration of the nucleic acid sequence administered by egg or oocyte is between 1 and 50 ng per μl, preferably between 5 and 40 ng per μl.

10) Method according to any one of claims 7 to 9, characterised in that the said exogenous nucleic acid sequence comprises a sequence of interest framed by two distinct nucleic acid sequences, the said distinct sequences having homology with the nucleic acid sequences located upstream and downstream respectively of the recognition site for the endonuclease that is present in the genomic DNA.

11) Method according to any one of the preceding claims, characterised in that the non-human vertebrate egg or oocyte is an egg or oocyte of mammals, reptiles, amphibians, birds, insects or fish.

12) Method according to claim 11, characterised in that the non-human vertebrate egg or oocyte is a fish egg or oocyte chosen from the group comprising salmon, trout, tuna, halibut, catfish, zebrafish, Medaka, carp, stickleback, astyanax, tilapia, redfish, bass, sturgeon or loach.

13) Method according to any one of the preceding claims, characterised in that it also comprises a step of culturing the previously fertilised ooctye or the egg having a targeted genome modification under adapted conditions for allowing the development of the non-human vertebrate.

14) Method according to claim 13, characterised in that it also comprises a step prior to the culture step which corresponds to an incubation of the egg or ooctye at a temperature of 50 to 20° C. below the culture temperature, preferably 10° to 15° C. below, and for a length of time making it possible to maintain viability of the eggs greater than 5%, corresponding to the eggs arriving at hatching, preferably greater than 10%.

15) Method according to claim 14, characterised in that the said step prior to the culture step corresponds to an incubation carried out for a time between 1 and 24 hours, preferably between 1 and 20 hours.
